(19) United States
(12) Patent Application Publication
(10) Pub. No.: US 2010/0061960 A1
(43) Pub. Date: Mar. 11, 2010

(54) Amido Anti-Viral Compounds, Compositions, And Methods Of Use

(76) Inventors: Franz Ulrich Schmitz, Mill Valley, CA (US); Roopa Rai, San Carlos, CA (US); Ronald Griffith, Escondldo, CA (US); Christopher Don Roberts, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/504,184

(22) Filed: Jul. 16, 2009

Related U.S. Application Data
(60) Provisional application No. 61/082,063, filed on Jul. 18, 2008.

(51) Int. Cl. A61K 38/21; C07D 277/20; A61K 31/426; C07D 417/14; A61K 31/5377; C07D 413/14; A61P 31/12

(52) U.S. Cl. 424/85.4; 548/195; 514/371; 544/133; 514/236.8; 544/82; 514/232.2

(57) ABSTRACT

Disclosed are compounds, stereoisomers, tautomers, or pharmaceutically acceptable salts thereof, their preparation, use, and compositions thereof for treating an infection mediated at least in part by a virus in the Flaviviridae family of viruses.

### CROSS-REFERENCE TO RELATED PATENTS AND PATENT APPLICATIONS

This application is related to and claims the priority benefit of U.S. Provisional Patent Application Ser. No. 61/082,063 filed Jul. 18, 2008, which is incorporated by reference herein in its entirety.

### FIELD OF THE INVENTION

The invention relates to the field of pharmaceutical chemistry, in particular to compounds, their preparation, compositions, and uses thereof for treating viral infections in patients mediated, at least in part, by a virus in the Flaviviridae family of viruses.

### REFERENCES

The following publications are cited in this application as superscript numbers:
1. Szabo, et al., Pathol. Oncol. Res. 2003, 9:215-221.
2. Hoofnagle J H, Hepatology 1997, 26:15S-20S.
3. Thomson B J and Finch R G, Clin Microbial Infect. 2005, 11:86-94.
4. Moriishi K and Matsuura Y, Antivir. Chem. Chemother. 2003, 14:285-297.
5. Fried, et al. N. Engl. J Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, et al., Ann. Rep. Med. Chem 39, 223-237, 2004.
9. Watashi, et al, Molecular Cell, 19, 111-122, 2005
10. Horsmans, et al, Hepatology, 42, 724-731, 2005

All of the above publications are herein incorporated by reference in their entirety to the same extent as if each individual publication was specifically and individually indicated to be incorporated by reference in its entirety.

### BACKGROUND OF THE INVENTION

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursuit to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4A protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

Notwithstanding the above, the discovery of new compounds active against one or more members of the Flaviviridae family of viruses would be beneficial particularly in view of the difficulty currently faced in treating diseases mediated, at least in part, by one or more of such viruses.

### SUMMARY OF THE INVENTION

This invention is directed to compounds, their preparation, compositions, and uses thereof for treating viral infections mediated, at least in part, by a virus in the Flaviviridae family of viruses. In one embodiment, provided is compound of Formula (I) or a stereoisomer, tautomer, or pharmaceutically acceptable salt thereof, wherein:

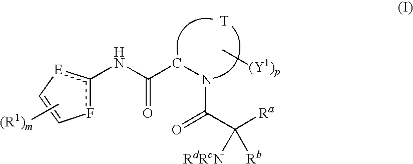

one of E or F is —N=and the other of E or F is —U—, —S—, or —NH—;

each R¹ is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, substituted sulfonyl, aminosulfonyl, and substituted alkylthio;

m is 1 or 2;

T is C₂-C₆ alkylene wherein optionally one —CH₂— group is replaced with —NR^{k}—, —S—, —SO—, —SO₂—, or —O—, and R^{k} is selected from the group consisting of hydrogen, acyl, aminocarbonyl, alkyl, substituted alkyl, substituted sulfonyl, and carboxyl ester;

Y¹ is attached to a carbon atom on T and is independently selected from the group consisting of halo, oxo, hydroxy, and alkoxy;

p is 0, 1, or 2;

R^{a} and R^{b} are independently selected from the group consisting of hydrogen, cyano, haloalkyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic;

R^{c} is selected from the group consisting of hydrogen, alkyl, and substituted alkyl; and

R^{d} is selected from the group consisting of hydrogen, alkyl, substituted alkyl, acyl, an amino acid residue attached via a peptide bond, aminocarbonyl, substituted aminocarbonyl, substituted sulfonyl, aminosulfonyl, carboxyl ester, cycloalkyl, substituted cycloalkyl, aryl, substituted aryl, heteroaryl, substituted heteroaryl, and heterocyclic.

In one embodiment provided is a pharmaceutical composition comprising a pharmaceutically acceptable carrier and a therapeutically effective amount of a compound of Formula (I) or a stereoisomer, tautomer, or pharmaceutically acceptable salt thereof.

In one embodiment provided is a method for treating a viral infection in a patient mediated at least in part by a virus in the Flaviviridae family of viruses, comprising administering to said patient a compound of Formula (I) or a stereoisomer, tautomer, or pharmaceutically acceptable salt thereof. In some aspects, the viral infection is mediated by hepatitis C virus.

### DETAILED DESCRIPTION

### Definitions

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C=C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO3H, substituted sulfonyl, sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to an acetylenic carbon atom.

“C₂-C₆ alkylene” refers to divalent straight chain alkyl groups having from 1 to 6 carbons.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O) substituted alkyl, —NR⁴⁷C(O)cycloalkyl, —NR⁴⁷C(O)substituted cycloalkyl, —NR⁴⁷C(O) cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O) heterocyclic, and —NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(0)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkeny —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(=NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(0)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O-C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring (e.g. fluorenyl). Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused, bridged, and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻¹).

“Spiro ring systems” refers to bicyclic ring systems that have only a single ring atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Sulfonyloxy” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Metabolite” refers to any derivative produced in a subject after administration of a parent compound. The metabolite may be produced from the parent compound by various biochemical transformations in the subject such as, for example, oxidation, reduction, hydrolysis, or conjugation. Metabolites include, for example, oxides and demethylated derivatives.

“Prodrug” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate [see Stahl and Wermuth, eds., “Handbook of Pharmaceutically Acceptable Salts”, (2002), Verlag Helvetica Chimica Acta, Zürich, Switzerland, for an extensive discussion of pharmaceutical salts, their selection, preparation, and use].

“Therapeutically effective amount” is an amount sufficient to treat a specified disorder or disease.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)—O—C(O)—.

It is understood that in all substituted groups defined above, polymers arrived at by defining substituents with further substituents to themselves are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of Formula (I) or a stereoisomer, tautomer, or pharmaceutically acceptable salt thereof, wherein:

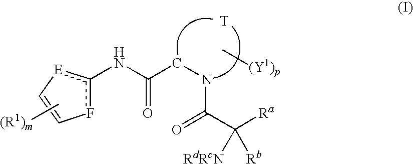

one of E or F is —N═ and the other of E or F is —O—, —S—, or —NH—;

each R¹ is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, substituted sulfonyl, aminosulfonyl, and substituted alkylthio;

m is 1 or 2;

T is C₂-C₆ alkylene wherein optionally one —CH₂— group is replaced with —NR^{k}—, —S—, —SO—, —SO₂—, or —O—, and R^{k} is selected from the group consisting of hydrogen, acyl, aminocarbonyl, alkyl, substituted alkyl, substituted sulfonyl, and carboxyl ester;

Y¹ is attached to a carbon atom on T and is independently selected from the group consisting of halo, oxo, hydroxy, and alkoxy;

p is 0, 1, or 2;

R^{a} and R^{b} are independently selected from the group consisting of hydrogen, cyano, haloalkyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic;

R^{c} is selected from the group consisting of hydrogen, alkyl, and substituted alkyl; and

R^{d} is selected from the group consisting of hydrogen, alkyl, substituted alkyl, acyl, an amino acid residue attached via a peptide bond, aminocarbonyl, substituted aminocarbonyl, substituted sulfonyl, aminosulfonyl, carboxyl ester, cycloalkyl, substituted cycloalkyl, aryl, substituted aryl, heteroaryl, substituted heteroaryl, and heterocyclic.

In one embodiment, provided is a compound of Formula (II) or a stereoisomer, tautomer, or pharmaceutically acceptable salt thereof, wherein:

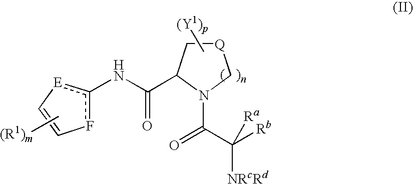

Q is selected from the group consisting of NR^{k}, S, SO, SO₂, O, and CH₂ optionally substituted with Y¹;

n is 1 or 2; and

E, F, R¹, m, R^{a}, R^{b}, R^{c}, R^{d}, Y¹, R^{k} and p are as defined for Formula (I).

In one embodiment, provided is a compound of Formula (III) or a stereoisomer, tautomer, pharmaceutically acceptable salt thereof, wherein:

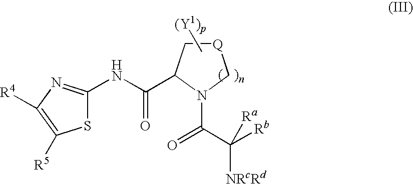

R⁴ is selected from the group consisting of cycloalkyl, substituted cycloalkyl, aryl, substituted aryl, heteroaryl, substituted heteroaryl, heterocyclic, and substituted heterocyclic;

R⁵ is independently selected from the group consisting of hydrogen, alkyl, substituted alkyl, and halo; and

R^{a}, R^{b}, R^{c}, R^{d}, Q, p, n, and Y¹ are as defined for Formula (II).

Various features relating to the Formulas and embodiments above are given below. These features when referring to different substitutents or variables can be combined with each other or with any other embodiments described in this application.

In some embodiments, at least one of R¹ or R⁴ is R²-L- wherein R² is selected from the group consisting of aryl, substituted aryl, heteroaryl, substituted heteroaryl, heterocyclic, and substituted heterocyclic; and L, defined in the R²-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R² is heterocyclic or substituted heterocyclic. In some aspects, L is a bond.

In some embodiments, R¹, R², or R⁴ is phenyl.

In some embodiments, R¹, R², or R⁴ is substituted phenyl. In some such aspects, said phenyl is substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio. In some aspects, at least one substitutent is cycloalkyl-C(O)NH—.

In some embodiments, R¹, R², or R⁴ is pyridine-2-yl or furan-2-yl.

In some embodiments, R¹, R², or R⁴ is phenyl substituted with at least one substituent selected from cyclopropyl-C(O)NH—, phenyl-C(O)NH—, cyclopentyl-C(O)NH—, 4-chlorophenyl-C(O)NH—, 4-chlorophenyl-C(O)NH—, methyl-C(O)NH—, methylamino, 4-methylphenyl-SO₂NH—, amino, ethyl-C(O)NH—, bromo, methoxy, methyl-SO₂NH—, chloro, phenyl-SO₂NH—, methyl-C(O)NH—, methyl-C(O)—, fluoro, methyl, ethyl, propyl, 4-fluorophenyl, nitro, phenyl, 4-bromobenzyloxy, cyclohexyl, isopropyl, tert-butyl, 4-methylpentyloxymethyl, NH₂C(O)—, hydroxy, cyclohexyl-NHC(O)CH₂S—, allyl, ethoxycarbonylmethylthio, dimethylamino, 3-nitro-phenyl, isobutyl, propoxy, butoxymethyl, butyl-C(O)NH—, methyl-NHC(O)—, ethyl-NHC(O)—, (2-oxo-hexahydro-thieno[3,4-d]imidazol-4-yl)-butyl-C(O)NH—, cyclopropyl-NHC(O)—, cyclohexyl-NHC(O)—, cyclopentyl-NHC(O)—, propyl, isobutyl, carboxy, pentyl-C(O)NH—, isopropyl-C(O)NH—, phenylamino-C(O)—, cylopropylamino-C(O)—, isopropylamino-C(O)—, ethylamino-C(O)—, and 2-(morpholin-4-yl)-ethylamino-C(O)—.

In some embodiments, R⁵ is H or methyl.

In some embodiments Q is NR^{k}, S, CH₂, or O.

In some embodiments, p is 0.

In some embodiments, R^{a} is selected from the group consisting of haloalkyl, alkyl, substituted alkyl, cycloalkyl, substituted cycloalkyl, aryl, substituted aryl, heteroaryl, substituted heteroaryl, heterocyclic, and substituted heterocyclic.

In some embodiments, R^{b} is H or methyl.

In yet other embodiments, the present invention provides a compound, stereoisomer, tautomer, or a pharmaceutically acceptable salt thereof selected from Table 1.

| Compound | Structure | Name |
|---|---|---|
| 101 | 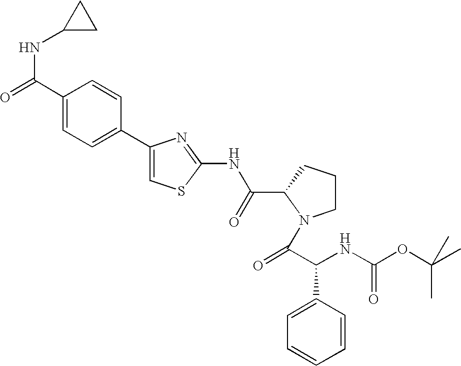 | ((R)-2-{(S)-2-[4-(4- Cyclopropylcarbamoyl-phenyl)- thiazol-2-ylcarbamoyl]-pyrrolidin-1- yl}-2-oxo-1-phenyl-ethyl)-carbamic acid tert-butyl ester |
| 102 | 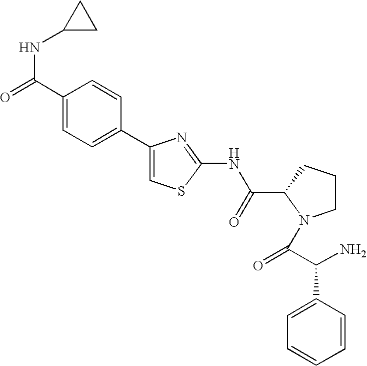 | (S)-1-((R)-2-Amino-2-phenyl- acetyl)-pyrrolidine-2-carboxylic acid [4-(4-cyclopropylcarbamoyl- phenyl)-thiazol-2-yl]-amide |
| 103 | 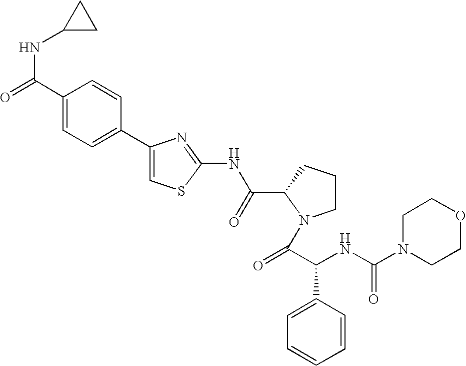 | Morpholine-4-carboxylic acid ((R)- 2-{(S)-2-[4-(4- cyclopropylcarbamoyl-phenyl)- thiazol-2-ylcarbamoyl]-pyrrolidin-1- yl}-2-oxo-1-phenyl-ethyl)-amide |
| 104 | 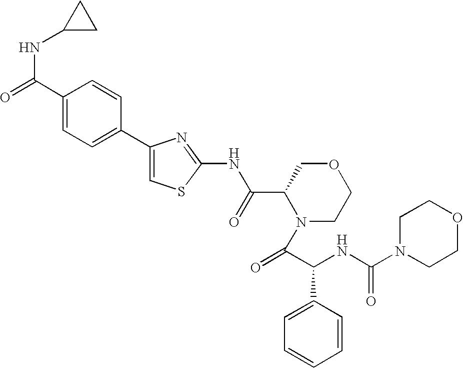 | (S)-4-{(R)-2-[(Morpholine-4- carbonyl)-amino]-2-phenyl-acetyl}- morpholine-3-carboxylic acid [4-(4- cyclopropylcarbamoyl-phenyl)- thiazol-2-yl]-amide |
| 105 | 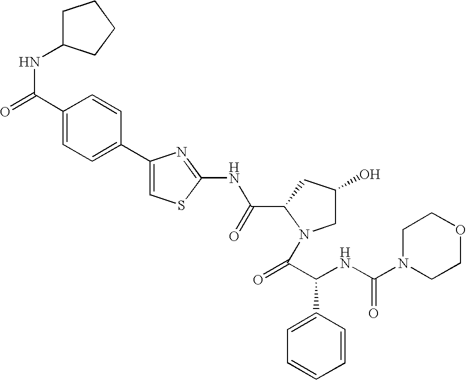 | Morpholine-4-carboxylic acid ((R)- 2-{(2S,4S)-2-[4-(4- cyclopentylcarbamoyl-phenyl)- thiazol-2-ylcarbamoyl]-4-hydroxy- pyrrolidin-1-yl}-2-oxo-1-phenyl- ethyl)-amide |
| 106 | 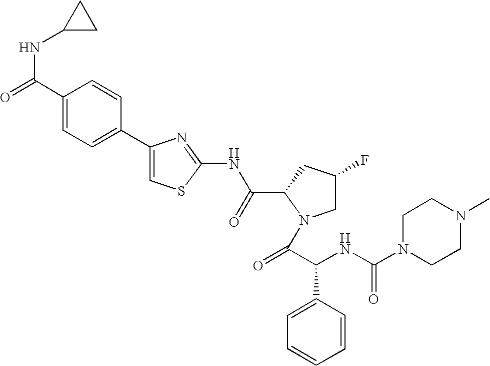 | 4-Methyl-piperazine-1-carboxylic acid ((R)-2-{(2S,4S)-2-[4-(4- cyclopropylcarbamoyl-phenyl)- thiazol-2-ylcarbamoyl]-4-fluoro- pyrrolidin-1-yl}-2-oxo-1-phenyl- ethyl)-amide |
| 107 | 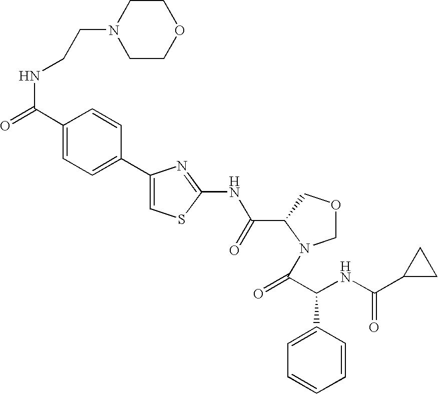 | (S)-3-[(R)-2- (Cyclopropanecarbonyl-amino)-2- phenyl-acetyl]-oxazolidine-4- carboxylic acid {4-[4-(2-morpholin- 4-yl-ethylcarbamoyl)-phenyl]- thiazol-2-yl}-amide |
| 108 | 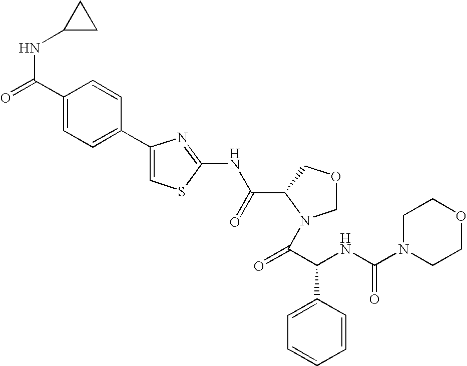 | Morpholine-4-carboxylic acid ((R)- 2-{(S)-2-[4-(4- cyclopropylcarbamoyl-phenyl)- thiazol-2-ylcarbamoyl]-oxazolidin- 3-yl}-2-oxo-1-phenyl-ethyl)-amide |
| 109 | 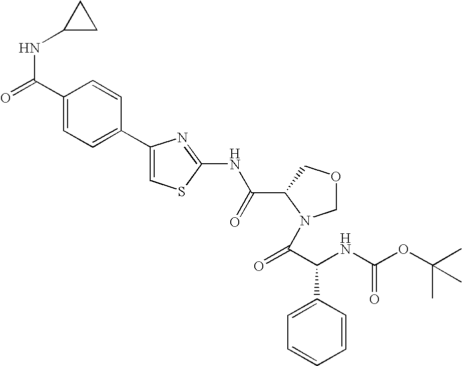 | ((R)-2- {(S)-4-[4-(4- Cyclopropylcarbamoyl-phenyl)- thiazol-2-ylcarbamoyl]-oxazolidin- 3-yl}-2-oxo-1-phenyl-ethyl)- carbamic acid tert-butyl ester |
| 110 | 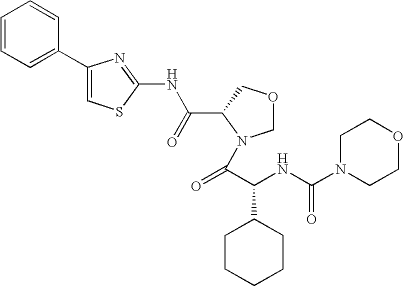 | Morpholine-4-carboxylic acid {(R)- 1-cyclohexyl-2-oxo-2-[(S)-4-(4- phenyl-thiazol-2-ylcarbamoyl)- oxazolidin-3-yl]-ethyl}-amide |
| 111 | 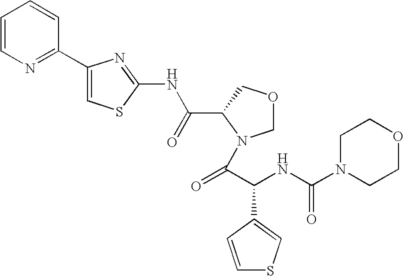 | Morpholine-4-carboxylic acid {(R)- 2-oxo-2-[(S)-4-(4-pyridin-2-yl- thiazol-2-ylcarbamoyl)-oxazolidin- 3-yl]-1-thiophen-3-yl-ethyl}-amide |
| 112 | 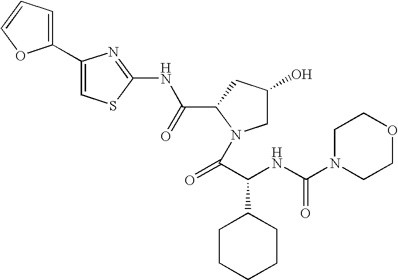 | Morpholine-4-carboxylic acid {(R)- 1-cyclohexyl-2-[(2S,4S)-2-(4-furan- 2-yl-thiazol-2-ylcarbamoyl)-4- hydroxy-pyrrolidin-1-yl]-2-oxo- ethyl}-amide |
| 113 | 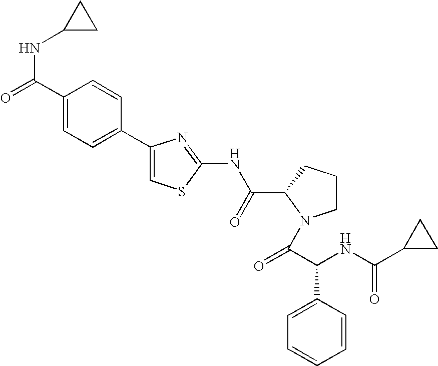 | (S)-1-[(R)-2- (Cyclopropanecarbonyl-amino)-2- phenyl-acetyl]-pyrrolidine-2- carboxylic acid [4-(4- cyclopropylcarbamoyl-phenyl)- thiazol-2-yl]-amide |
| 114 | 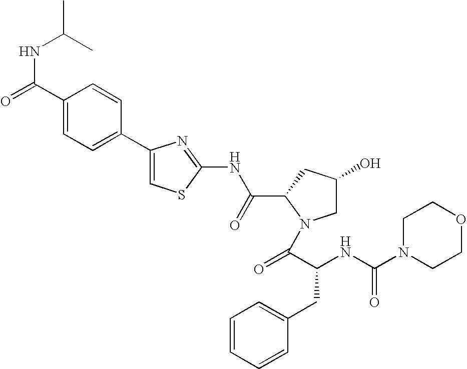 | Morpholine-4-carboxylic acid ((R)- 1-benzyl-2-{(2S,4S)-4-hydroxy-2- [4-(4-isopropylcarbamoyl-phenyl)- thiazol-2-ylcarbamoyl]-pyrrolidin-1- yl}-2-oxo-ethyl)-amide |
| 115 | 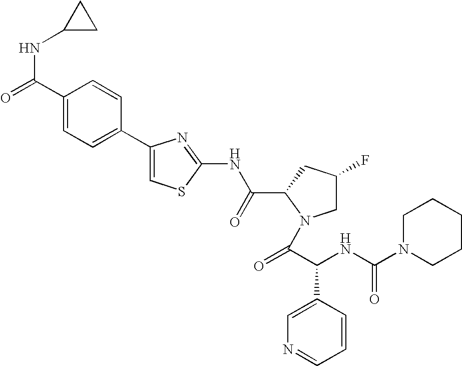 | Piperidine-1-carboxylic acid ((R)-2- {(2S,4S)-2-[4-(4- cyclopropylcarbamoyl-phenyl)- thiazol-2-ylcarbamoyl]-4-fluoro- pyrrolidin-1-yl}-2-oxo-1-pyridin-3- yl-ethyl)-amide |
| 116 | 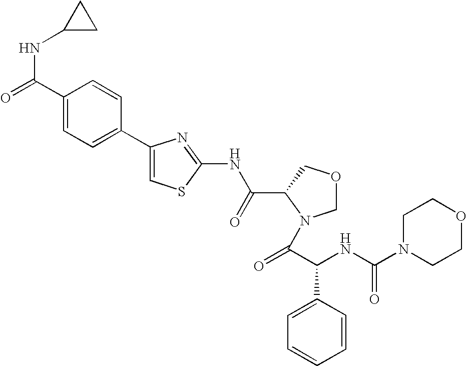 | Morpholine-4-carboxylic acid ((R)- 2-{(S)-4-[4-(4- cyclopropylcarbamoyl-phenyl)- thiazol-2-ylcarbamoyl]-oxazolidin- 3-yl}-2-oxo-1-phenyl-ethyl)-amide |
| 117 | 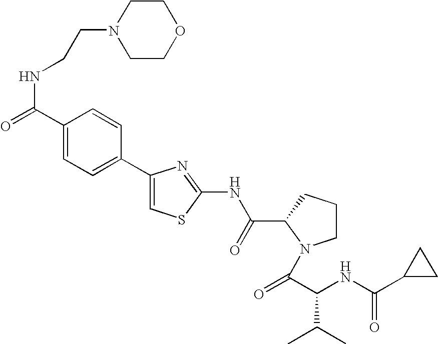 | (S)-1-[(R)-2- (Cyclopropanecarbonyl-amino)-3- methyl-butyryl]-pyrrolidine-2- carboxylic acid {4-[4-(2-morpholin- 4-yl-ethylcarbamoyl)-phenyl]- thiazol-2-yl}-amide |
| 118 | 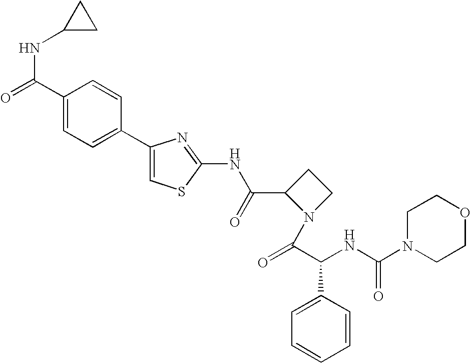 | Morpholine-4-carboxylic acid ((R)- 2-{2-[4-(4-cyclopropylcarbamoyl- phenyl)-thiazol-2-ylcarbamoyl]- azetidin-1-yl}-2-oxo-1-phenyl- ethyl)-amide |
| 119 | 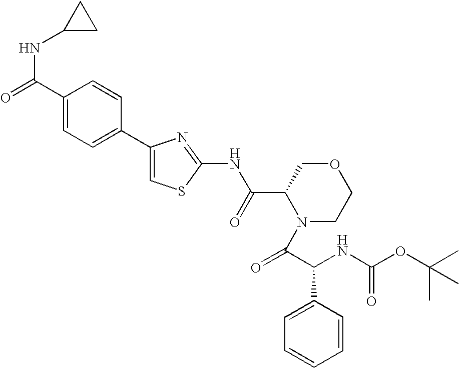 | ((R)-2-{(S)-3-[4-(4- Cyclopropylcarbamoyl-phenyl)- thiazol-2-ylcarbamoyl]-morpholin- 4-yl}-2-oxo-1-phenyl-ethyl)- carbamic acid tert-butyl ester |
| 120 | 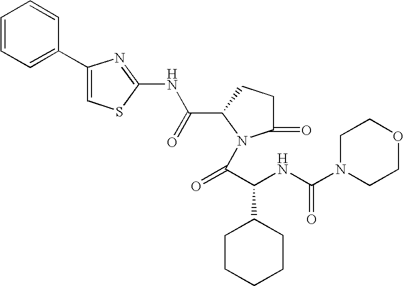 | Morpholine-4-carboxylic acid {(R)- 1-cyclohexyl-2-oxo-2-[(S)-2-oxo-5- (4-phenyl-thiazol-2-ylcarbamoyl)- pyrrolidin-1-yl]-ethyl}-amide |
| 121 | 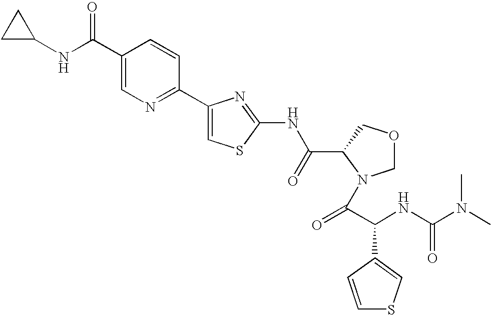 | N-Cyclopropyl-6-[2-({(S)-3-[(R)-2- (3,3-dimethyl-ureido)-2-thiophen-3- yl-acetyl]-oxazolidine-4-carbonyl}- amino)-thiazol-4-yl]-nicotinamide |
| 122 | 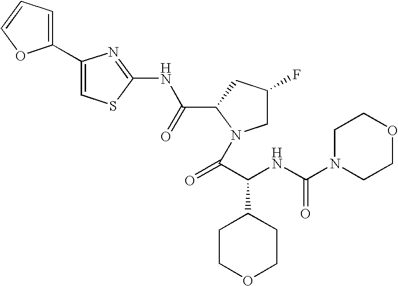 | Morpholine-4-carboxylic acid [(R)- 2-[(2S,4S)-4-fluoro-2-(4-furan-2-yl- thiazol-2-ylcarbamoyl)-pyrrolidin-1- yl]-2-oxo-1-(tetrahydro-pyran-4-yl)- ethyl]-amide |
| 123 | 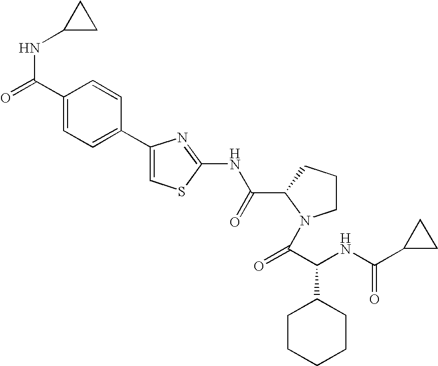 | (S)-1-[(R)-2-Cyclohexyl-2- (cyclopropanecarbonyl-amino)- acetyl]-pyrrolidine-2-carboxylic acid [4-(4-cyclopropylcarbamoyl- phenyl)-thiazol-2-yl]-amide |
| 124 | 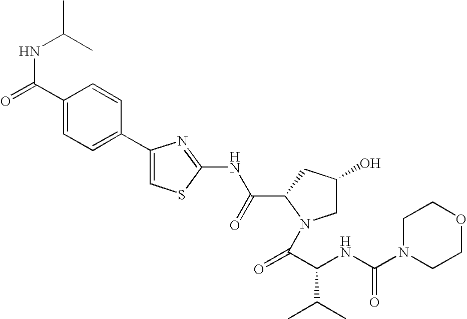 | Morpholine-4-carboxylic acid ((R)- 1-{(2S,4S)-4-hydroxy-2-[4-(4- isopropylcarbamoyl-phenyl)-thiazol- 2-ylcarbamoyl]-pyrrolidine-1- carbonyl}-2-methyl-propyl)-amide |
| 125 | 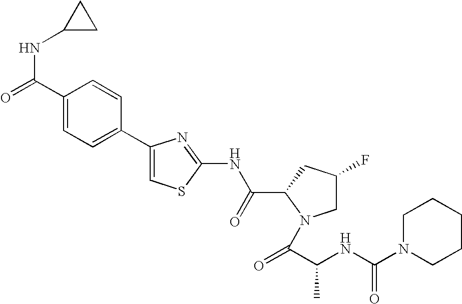 | Piperidine-1-carboxylic acid ((R)-2- {(2S,4S)-2-[4-(4- cyclopropylcarbamoyl-phenyl)- thiazol-2-ylcarbamoyl]-4-fluoro- pyrrolidin-1-yl}-1-methyl-2-oxo- ethyl)-amide |
| 126 | 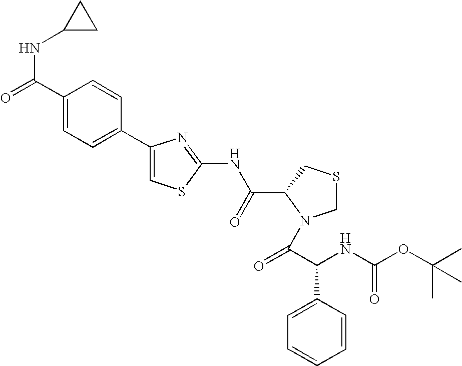 | ((R)-2-{(R)-4-[4-(4- Cyclopropylcarbamoyl-phenyl)- thiazol-2-ylcarbamoyl]-thiazolidin- 3-yl}-2-oxo-1-phenyl-ethyl)- carbamic acid tert-butyl ester |
| 127 | 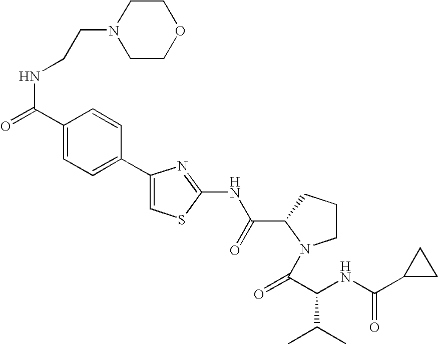 | (S)-1-[(R)-2- (Cyclopropanecarbonyl-amino)-3- methyl-butyryl]-pyrrolidine-2- carboxylic acid {4-[4-(2-morpholin- 4-yl-ethylcarbamoyl)-phenyl]- thiazol-2-yl}-amide |
| 128 | 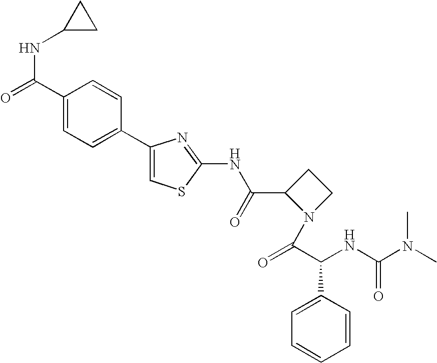 | 1-[(R)-2-(3,3-Dimethyl-ureido)-2- phenyl-acetyl]-azetidine-2- carboxylic acid [4-(4- cyclopropylcarbamoyl-phenyl)- thiazol-2-yl]-amide |
| 129 | 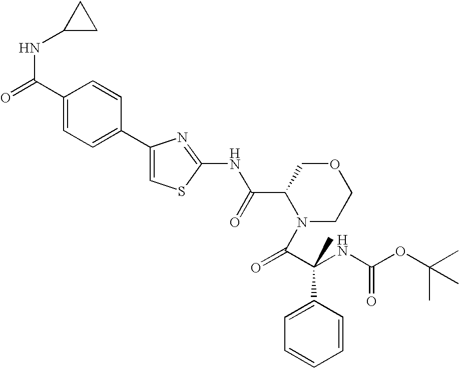 | ((R)-2-{(S)-3-[4-(4- Cyclopropylcarbamoyl-phenyl)- thiazol-2-ylcarbamoyl]-morpholin- 4-yl}-2-oxo-1-methyl-1-phenyl- ethyl)-carbamic acid tert-butyl ester |
| 130 | 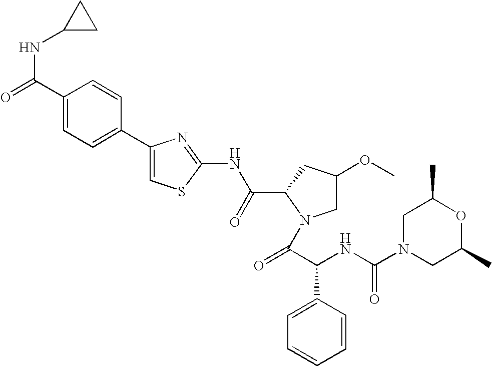 | (2R,6S)-2,6-Dimethyl-morpholine- 4-carboxylic acid ((R)-2-{(S)-2-[4- (4-cyclopropylcarbamoyl-phenyl)- thiazol-2-ylcarbamoyl]-4-methoxy- pyrrolidin-1-yl}-2-oxo-1-phenyl- ethyl)-amide |
| 131 | 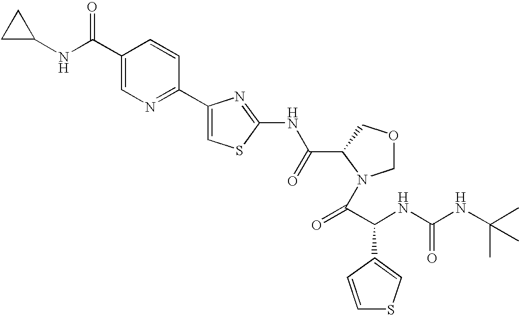 | 6-[2-({(S)-3-[(R)-2-(3-tert-Butyl- ureido)-2-thiophen-3-yl-acetyl]- oxazolidine-4-carbonyl}-amino)- thiazol-4-yl]-N-cyclopropyl- nicotinamide |
| 132 | 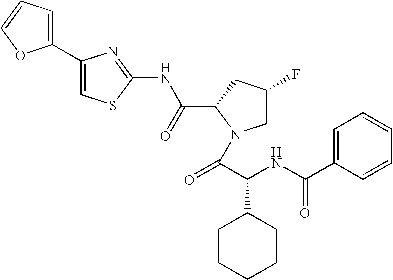 | (2S,4S)-1-((R)-2-Benzoylamino-2- cyclohexyl-acetyl)-4-fluoro- pyrrolidine-2-carboxylic acid (4- furan-2-yl-thiazol-2-yl)-amide |
| 133 | 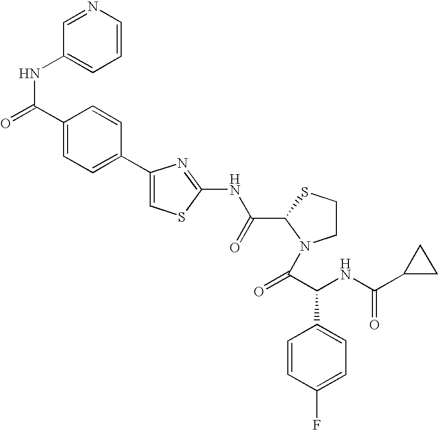 | (S)-3-[(R)-2- (Cyclopropanecarbonyl-amino)-2- (4-fluoro-phenyl)-acetyl]- thiazolidine-2-carboxylic acid {4-[4- (pyridin-3-ylcarbamoyl)-phenyl]- thiazol-2-yl}-amide |
| 134 | 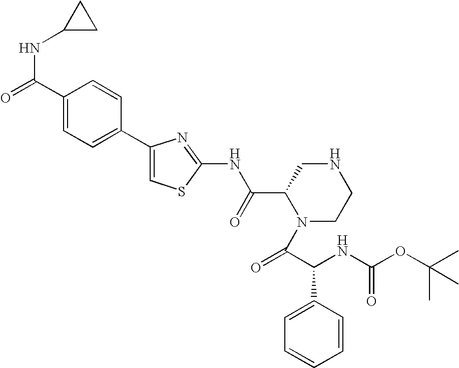 | ((R)-2-{(S)-2-[4-(4- Cyclopropylcarbamoyl-phenyl)- thiazol-2-ylcarbamoyl]-piperazin-1- yl}-2-oxo-1-phenyl-ethyl)-carbamic acid tert-butyl ester |
| 135 | 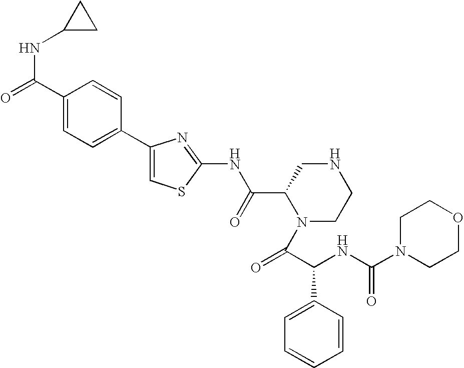 | Morpholine-4-carboxylic acid ((R)- 2-{(S)-2-[4-(4- cyclopropylcarbamoyl-phenyl)- thiazol-2-ylcarbamoyl]-piperazin-1- yl}-2-oxo-1-phenyl-ethyl)-amide |
| 136 | 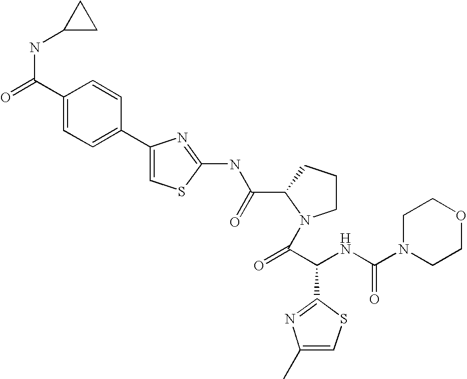 | Morpholine-4-carboxylic acid ((R)- 2-{(S)-2-[4-(4- cyclopropylcarbamoyl-phenyl)- thiazol-2-ylcarbamoyl]-pyrrolidin-1- yl}-1-(4-methyl-thiazol-2-yl)-2-oxo- ethyl]-amide |
| 137 | 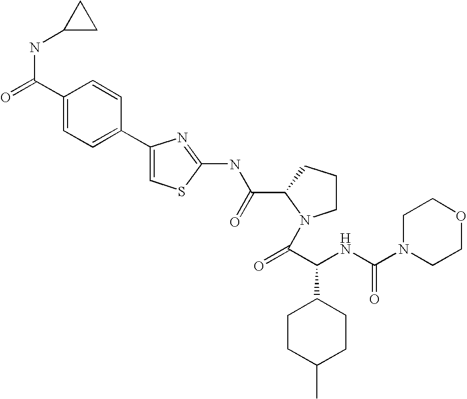 | Morpholine-4-carboxylic acid ((R)- 2-{(S)-2-[4-(4- cyclopropylcarbamoyl-phenyl)- thiazol-2-ylcarbamoyl]-pyrrolidin-1- yl}-1-(4-methyl-cyclohexyl)-2-oxo- ethyl]-amide |

In one embodiment provided is a pharmaceutical composition comprising a pharmaceutically acceptable carrier and a therapeutically effective amount of a compound, stereoisomer, tautomer, or pharmaceutically acceptable salt of any one of Formula (I)-(III) or of the compounds in Table 1. In another embodiment provided is a method for treating a viral infection in a patient mediated at least in part by a virus in the Flaviviridae family of viruses, comprising administering to said patient such compositions. In some aspects, the viral infection is mediated by hepatitis C virus.

In other aspects, the administration of a therapeutically effective amount of the compounds and/or compositions of the invention are used in combination with one or more agents active against hepatitis C virus. These agents include an inhibitor of HCV proteases, HCV polymerase, HCV helicase, HCV NS4B protein, HCV entry, HCV assembly, HCV egress, HCV NS5A protein, or inosine 5′-monophosphate dehydrogenase. In other embodiments, the agent is interferon.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of compounds of Formula (I)-(III) may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula (I)-(III) in combination with at least one pharmaceutically acceptable excipient. Acceptable excipients are non-toxic, aid administration, and do not adversely affect the therapeutic benefit of the compound of Formula (I)-(III). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula (I)-(III) based on the total formulation, with the balance being one or more suitable pharmaceutical excipients. Preferably, the compound is present at a level of about 1-80 wt %. Representative pharmaceutical formulations containing a compound of Formula (I)-(III) are described below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV.

References herein to agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of Formula (I)-(III) and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of Formula (I)-(III) and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

A variety of amide coupling reagents may be used to from the amide bond, including the use of carbodiimides such as N-N′-dicyclohexylcarbodiimide (DCC), N-N′-diisopropylcarbodiimide (DIPCDI), and 1-ethyl-3-(3′-dimethylaminopropyl)carbodiimide (EDCI). The carbodiimides may be used in conjunction with additives such as benzotriazoles 7-aza-1-hydroxybenzotriazole (HOAt), 1-hydroxybenzotriazole (HOBt), and 6-chloro-1-hydroxybenzotriazole (Cl-HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP).

The amide formation step may be conducted in a polar solvent such as dimethylformamide (DMF) and may also include an organic base such as diisopropylethylamine (DIPEA).

Scheme 1 shows a general synthesis of the compounds wherein for illustrative purposes T is —CH₂CH₂CH₂— and m is 1. Bromide 1.1 is reacted with thiourea to form amine 1.2. Coupling of amine 1.2 with t-butoxycarbonyl-proline using standard amide coupling procedures forms amide 1.3 that is then treated with an acid such as trifluoroacetic acid to give amine 1.4. A second amide coupling with acid 1.5 gives the desired compound 1.6.

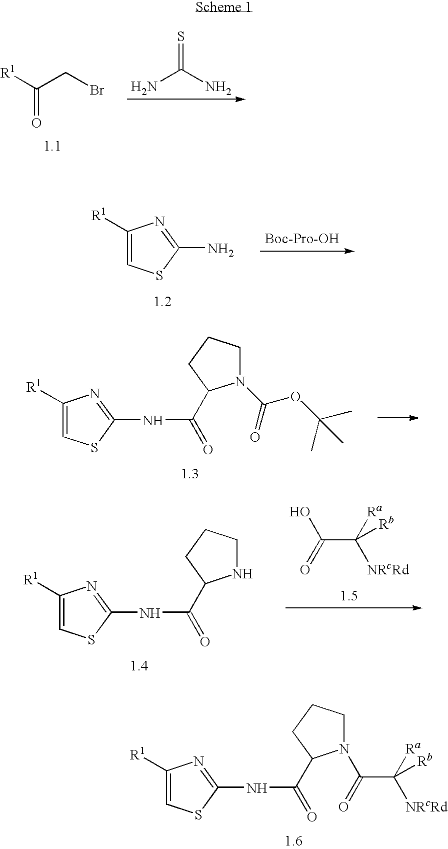

Scheme 2 shows the synthesis of compounds wherein for illustrative purposes T is —CH₂CH₂CH₂— and R¹ is substituted phenyl. The final product is prepared by sequential amide couplings using the indicated starting materials and employing the necessary deprotection steps. Details of the synthesis of the final product are given in Examples 1-3.

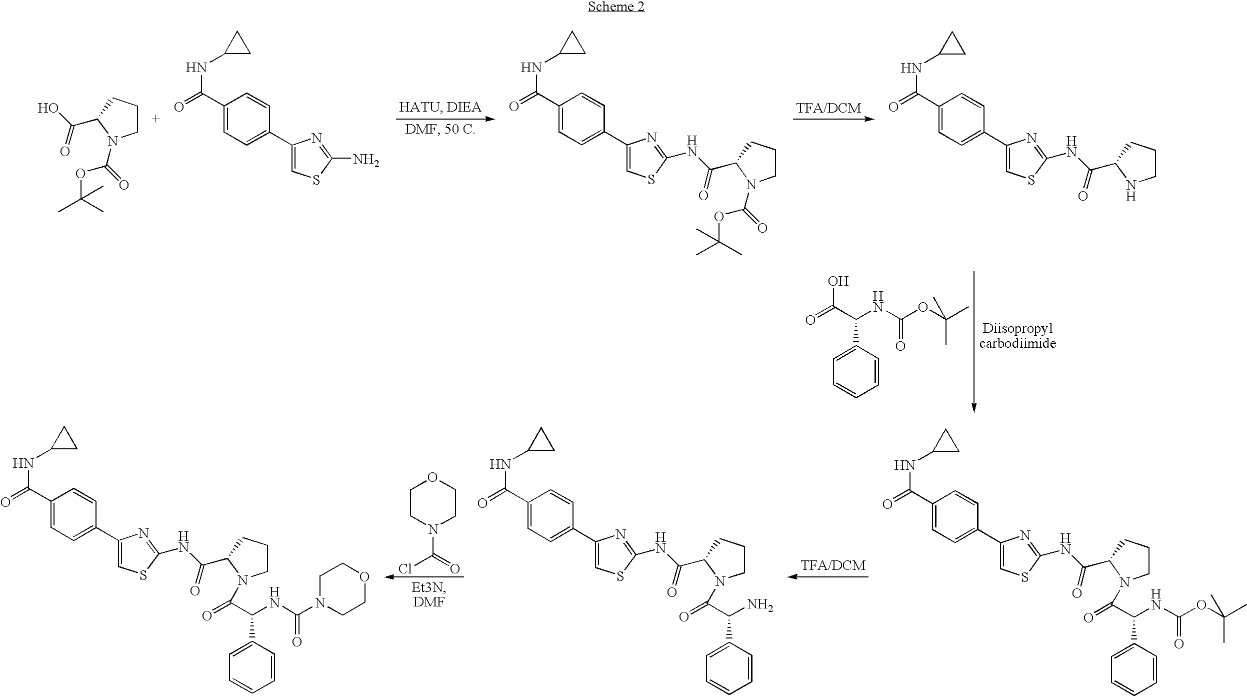

Scheme 3 shows the synthesis of the compounds wherein for illustrative purposes T is —CH₂OCH₂CH₂— and R¹ is substituted phenyl. The final product is prepared by sequential amide couplings using the indicated starting materials and employing the necessary deprotection steps. Details of the synthesis of the final product are given in Example 4.

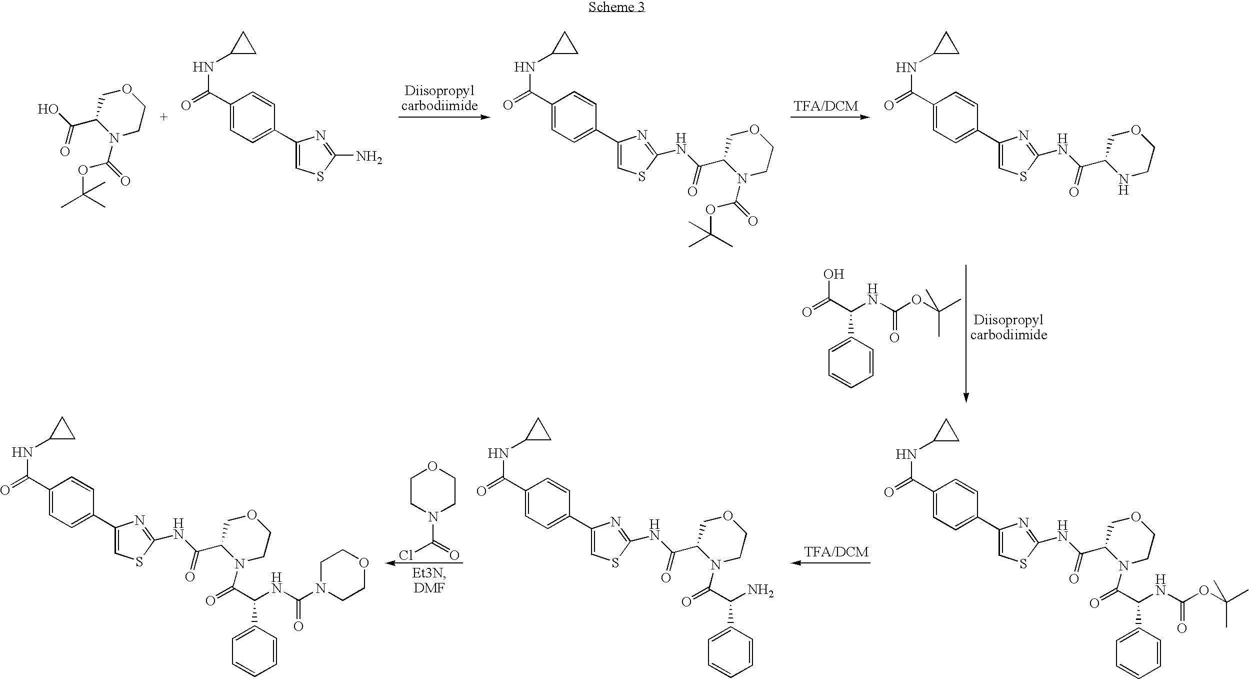

The forgoing and other aspects of the present invention may be better understood in connection with the following representative examples.

### Synthetic Examples

In the examples below and the synthetic schemes above, the following abbreviations have the following meanings If an abbreviation is not defined, it has its generally accepted meaning.
aq. aqueous
μL microliters
μM micromolar
NMR nuclear magnetic resonance
br broad
d doublet
δ chemical shift
° C. degrees celcius
DCM dichloromethane
dd doublet of doublets
DMEM Dulbeco's Modified Eagle's Medium
DMF N,N-dimethylformamide
DMSO dimethylsulfoxide
EtOAc ethyl acetate
g gram
h or hr hours
HCV hepatitus C virus
HPLC high performance liquid chromatography
Hz hertz
IU International Units
IC₅₀ inhibitory concentration at 50% inhibition
J coupling constant (given in Hz unless otherwise indicated)
m multiplet
M molar
M+H⁺ parent mass spectrum peak plus H⁺
mg milligram
mL milliliter
mM millimolar
mmol millimole
MS mass spectrum
nm nanomolar
ppm parts per million
q.s. sufficient amount
s singlet
sat. saturated
t triplet
TFA trifluoroacetic acid

### EXAMPLE 1

### ((R)-2-{(S)-2-[4-(4-Cyclopropylcarbamoyl-phenyl)-thiazol-2-ylcarbamoyl]pyrrolidin-1-yl}-2-oxo-1-phenyl-ethyl)-carbamic acid 1,1-dimethyl-propyl ester (Compound 101)

Step 1: (S)-2-[4-(4-Cyclopropylcarbamoyl-phenyl)-thiazol-2-ylcarbamoyl]pyrrolidine-1-carboxylic acid tert-butyl ester

BOC-proline (2.0 g, 9.3 mmol) was combined with HATU (3.5 g, 9.3 mmol) in 12 mL of DMF. To this solution was added DIEA (5.8 mL, 32.5 mmol) and the resulting solution was stirred at room temperature for 15 minutes. Thiazole amine (2.6 g, 9.3 mmol) was added to the mixture which was heated at 50° C. overnight. Reaction mixture was diluted using EtOAc and extracted with sat.NaHCO₃ (aq). Organic phase was isolated, washed with H₂O, dried over MgSO₄, and evaporated to yield the crude desired product as a yellow solid. The crude was further purified using silica gel chromatography, eluted using 3:1 hexane/ethyl acetate solvent system. (2.4 g)

Step 2: Pyrrolidine-2-carboxylic acid [4-(4-cyclopropylcarbamoyl-phenyl)-thiazol-2-yl]-amide)

(S)-2-[4-(4-Cyclopropylcarbamoyl-phenyl)-thiazol-2-ylcarbamoyl]-pyrrolidine-1-carboxylic acid tert-butyl ester (2.4 g, 5.2 mmol) was dissolved in 20 mL of 3:1 solution of DCM/TFA. This solution was stirred at room temperature for 1 hour and the evaporated to dryness. In order to neutralize the TFA salt, the resulting oil was redissolved in EtOAc and extracted with sat.NaHCO₃ (aq). Organic phase was isolated, dried over MgSO₄, and evaporated to yield the desired product as a white solid.

Step 3: ((R)-2-{(S)2-[4-(4-Cyclopropylcarbamoyl-phenyl)-thiazol-2-ylcarbamoyl]-pyrrolidin-1-yl}-2-oxo-1-phenyl-ethyl)-carbamic acid tert-butyl ester (compound 101)

BOC-D-Phg-OH, (48.2 mg, 0.19 mmol) was combined with pyrrolidine-2-carboxylic acid [4-(4-cyclopropylcarbamoyl-phenyl)-thiazol-2-yl]-amide), free amine, (59 mg, 0.16 mmol) in 3 mL of THF. This solution was stirred at room temperature for 5 minutes and then DIPC (0.03 mL, 1.9 mmol) was added. Reaction mixture was stirred at room temperature for 18 hours.

It was diluted using EtOAc and extracted with sat.NaHCO₃ (aq). Organic phase was isolated, washed with H₂O, dried over MgSO₄, and evaporated to yield the crude desired product. The crude material was purified using reverse phase HPLC. MS: 590.0 (M+H⁺); H¹ NMR (DMSO-d₆): δ(ppm) 12.33 (s, 1H), 8.42 (d, 1H), 7.86 (q, 4H), 7.76 (s, 1H), 7.32 (m, 5H), 5.46 (d, 1H), 4.72 (br s, NH, 1H), 4.48 (m, 2H), 3.80 (m, 1H), 3.10 (m, 1H), 2.84 (m, 1H), 2.05-1.75 (m, 4H), 1.34 (s, 9H), 0.66 (m, 2H), 0.56 (m, 2H)

### EXAMPLE 2

### (S)-1-((R)-2-Amino-2-phenyl-acetyl)-pyrrolidine-2-carboxylic acid [4-(4-cyclopropylcarbamoyl-phenyl)-thiazol-2-yl]-amide (Compound 102)

((R)-2-{(S)-2-[4-(4-Cyclopropylcarbamoyl-phenyl)-thiazol-2-ylcarbamoyl]-pyrrolidin-1-yl}-2-oxo-1-phenyl-ethyl)-carbamic acid tert-butyl ester (compound 101, 67 mg) was dissolved in 5 mL of 3:1 solution of DCM/TFA. This solution was stirred at room temperature for 1 hour and the evaporated to dryness. In order to neutralize the TFA salt, the resulting oil was redissolved in EtOAc and extracted with sat.NaHCO₃ (aq). Organic phase was isolated, dried over MgSO₄, and evaporated to yield the desired product as a white solid. (˜30% racemized). MS: 590.0 (M+H⁺); H¹ NMR (TFA salt) (DMSO-d₆): δ(ppm) 12.54 (s, 1H), 8.60 (br s, 2H), 8.43 (d, 2H), 7.88 (q, 4H), 7.77 (s, 1H), 7.48 (br s, 5H), 5.45 (br s, 1H), 4.60 (d, 1H), 3.81 (m, 1H), 2.85 (m, 1H), 2.76 (m, 1H), 2.03−1.75 (m, 4H), 1.27 (m, 1H), 0.67 (m, 2H), 0.57 (m, 2H).

### EXAMPLE 3

### Morpholine-4-carboxylic acid ((R)-2-{(S)-2-[4(4-cyclopropylcarbamoyl-phenyl)-thiazol-2-ylcarbamoyl]-ppyrrolidin-1-yl}-2-oxo-1-phenyl-ethyl)-amide (Compound 103)

(S)-1-((R)-2-Amino-2-phenyl-acetyl)-pyrrolidine-2-carboxylic acid [4-(4-cyclopropylcarbamoyl-phenyl)-thiazol-2-yl]-amide (Compound 102, 80 mg, 0.16 mmol) was dissolved in 3 mL of dichloromethane. To this solution was added triethylamine (0.0272 mL, 0.2 mmol), followed by morpholinocarbonyl chloride (0.023 mL, 0.2 mmol). Reaction mixture was stirred at room temperature for 30 minutes. Reaction mixture was quenched using H₂O. Organic phase was isolated, dried and evaporated. The crude material was purified using reverse phase HPLC. MS: 603.1 (M+H⁺); H¹ NMR (DMSO-d₆): δ(ppm) 12.12 (s, 1H), 8.42 (d, 1H), 7.87 (q, 4H), 7.76 (s, 1H), 7.35 (m, 5H), 5.56 (m, 1H), 4.53 (dd, 1H), 3.87 (m, 1H), 3.46 (m, 4H), 3.30 (m, 4H), 3.18 (m, 1H), 2.83 (m, 1H), 2.07-1.78 (m, 4H), 0.67 (m, 2H), 0.56 (m, 2H).

### EXAMPLE 4

### (S)-4-{(R)-2-[(Morpholine-4-carbonyl)-amino]-2-phenyl-acetyl}-morpholine-3-carboxylic acid [4-(4-cyclopropylcarbamoyl-phenyl)-thiazol-2-yl]amide (Compound 104)

(S)-Morpholine-3,4-dicarboxylic acid 4-tert-butyl ester (0.52 gm, 2.25 mmol), 4-(2-amino-thiazol-4-yl)-N-cyclopropyl-benzamide (0.49 g, 1.89 mmol) and diisopropyl carbodiimide (0.44 mL, 2.8 mmol) were dissolved in dichloromethane (DCM, 9 mL) and acetonitrile (5 mL) and the mixture heated at 50° C. for 4 h and 80° C. for 2 h. The mixture was concentrated and 15 mL DCM added. The undissolved solid urea side product was filtered off and the filtrate was purified by silica gel chromatography to afford 0.53 gm of (S)-3-[4-(4-Cyclopropylcarbamoyl-phenyl)-thiazol-2-ylcarbamoyl]-morpholine-4-carboxylic acid tert-butyl ester as a white solid.

The remaining steps followed procedures identical to those described for the synthesis of morpholine-4-carboxylic acid ((R)-2-{(S)-2-[4-(4-cyclopropylcarbamoyl-phenyl)-thiazol-2-ylcarbamoyl]-pyrrolidin-1-yl}-2-oxo-1-phenyl-ethyl)-amide (compound 103, Example 2 and 3). The synthesis afforded (S)-4-{(R)-2-[(morpholine-4-carbonyl)-amino]-2-phenyl-acetyl}-morpholine-3-carboxylic acid [4-(4-cyclopropylcarbamoyl-phenyl)-thiazol-2-yl]-amide: LCMS 619.1 (M+H⁺); H¹ NMR (CDCl₃): δ(ppm) 7.82 (m, 4H), 7.43 (m, 5H), 7.23 (s, 1H), 6.34 (br s, 1H), 5.76 (m, 2H), 5.36 (d, 1H, J=3.3 Hz), 4.72 (d, 1H, J=12 Hz), 3.72-2.81 (m under br water peak), 0.90 (m, 2H), 0.67 (d, 2H).

### Biological Examples

### Example 1

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al. J. of Vir., 73:1649-1654, 1999; Ishii et al., Hepatology, 29:1227-1235, 1999; Lohmann et al., J. of Bio. Chem., 274:10807-10815, 1999; and Yamashita et al., J. of Bio. Chem., 273:15479-15486, 1998.

### Replicon Assay

A cell line, ET (Huh-lucubineo-ET) is used for screening of compounds of the present invention for inhibiting HCV replication. The ET cell line is stably transfected with RNA transcripts harboring a I₃₈₉luc-ubi-neo/NS3-3′ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells are grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They are all available through Life Technologies (Bethesda, Md.). The cells are plated at 0.5-1.0×10⁴ cells/well in the 96 well plates and incubated for 24 hrs before adding testing compounds. Then the compounds are added to the cells to achieve a final concentration of 0.1 nM to 50 μM and a final DMSO concentration of 0.5%. Luciferase activity is measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication is plotted relative to no compound control. Under the same condition, cytotoxicity of the compounds is determined using cell proliferation reagent, WST-1(Roche, Germany). The compounds showing antiviral activities, but no significant cytotoxicities are chosen to determine EC₅₀ and TC₅₀. For these determinations, a 10 point 2-fold serial dilution for each compound is used, which spans a concentration range of 1000 fold. EC₅₀ and similarly TC₅₀ values are calculated by fitting % inhibition at each concentration to the following equation:

% inhibition=100%/[(EC₅₀/[I])^{b}+1]

where b is Hill's coefficient.

When tested at 100 μM, the compounds of this invention will exhibit a % inhibition of at least 30%, preferably at least 80%. More preferably, when tested at 50 μM the compounds will exhibit a % inhibition of at least 30%, preferably at least 80%. Still more preferably when tested at 10 μM the compounds will exhibit a % inhibition of at least 30%, preferably at least 80%.

### Formulation Examples

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound | 200 |
| Lactose, spray-dried | 148 |
| Magnesium stearate | 2 |

### Example 3

### Suspension Formulation

The following ingredients are mixed to form a suspension for oral administration.

| Ingredient | Amount | |
|---|---|---|
| Compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co.) | 1.0 | g |
| flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg |
|---|---|
| Compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound | 500 mg |
| Witepsol ® H-15 | balance |

What is claimed is:

1. A compound that is Formula (I) or a stereoisomer, tautomer, or pharmaceutically acceptable salt thereof, wherein: 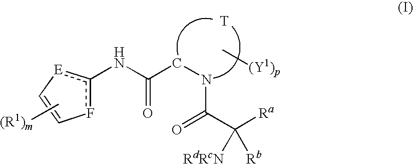
one of E or F is —N═ and the other of E or F is —O—, —S—, or —NH—;
each R¹ is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, substituted sulfonyl, aminosulfonyl, and substituted alkylthio;
m is 1 or 2;
T is C₂-C₆ alkylene wherein optionally one —CH₂-group is replaced with —NR^{k}—, —S—, —SO—, —SO₂—, or —O—, and R^{k} is selected from the group consisting of hydrogen, acyl, aminocarbonyl, alkyl, substituted alkyl, substituted sulfonyl, and carboxyl ester;
Y¹ is attached to a carbon atom on T and is independently selected from the group consisting of halo, oxo, hydroxy, and alkoxy;
p is 0, 1, or 2;
R^{a} and R^{b} are independently selected from the group consisting of hydrogen, cyano, haloalkyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic;
R^{c} is selected from the group consisting of hydrogen, alkyl, and substituted alkyl; and
R^{d} is selected from the group consisting of hydrogen, alkyl, substituted alkyl, acyl, an amino acid residue attached via a peptide bond, aminocarbonyl, substituted aminocarbonyl, substituted sulfonyl, aminosulfonyl, carboxyl ester, cycloalkyl, substituted cycloalkyl, aryl, substituted aryl, heteroaryl, substituted heteroaryl, and heterocyclic.

2. A compound of claim 1 that is Formula (II) or a stereoisomer, tautomer, or pharmaceutically acceptable salt thereof, wherein: 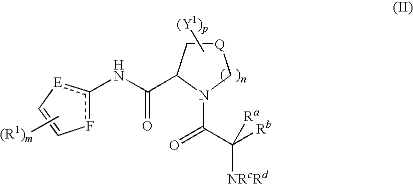
Q is selected from the group consisting of NR^{k}, S, SO, SO₂, O, and CH₂ optionally substituted with Y¹;
n is 1 or 2; and
E, F, R¹, m, R^{a}, R^{b}, R^{c}, R^{d}, Y¹, R^{k} and p are as defined for Formula (I).

3. A compound of claim 2 wherein at least one of R¹ is R²—L— wherein R² is selected from the group consisting of aryl, substituted aryl, heteroaryl, substituted heteroaryl, heterocyclic, and substituted heterocyclic; and L, defined in the R²-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O—provided that when L is ═CH—, R² is heterocyclic or substituted heterocyclic.

4. A compound of claim 3 wherein L is a bond.

5. A compound of claim 3 wherein R² is substituted phenyl.

6. A compound of claim 2 wherein Q is NR^{k}, S, CH₂, or O.

7. A compound of claim 2 wherein p is 0.

8. A compound of claim 2 wherein R^{a} is selected from the group consisting of haloalkyl, alkyl, substituted alkyl, cycloalkyl, substituted cycloalkyl, aryl, substituted aryl, heteroaryl, substituted heteroaryl, heterocyclic, and substituted heterocyclic.

9. A compound of claim 2 wherein R^{b} is H or methyl.

10. A compound of claim 1 having Formula (III) or a stereoisomer, tautomer, pharmaceutically acceptable salt thereof, wherein: 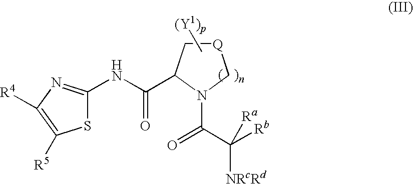
R⁴ is selected from the group consisting of cycloalkyl, substituted cycloalkyl, aryl, substituted aryl, heteroaryl, substituted heteroaryl, heterocyclic, and substituted heterocyclic;
R⁵ is independently selected from the group consisting of hydrogen, alkyl, substituted alkyl, and halo; and R^{a}, R^{b}, R^{c}, R^{d}, Q, p, n, and Y¹ are as defined for Formula (II).

11. A compound of claim 10 wherein R⁴ is substituted phenyl.

12. A compound of claim 11 wherein R⁴ is phenyl substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio.

13. A compound of claim 10 wherein Q is NR^{k}, S, CH₂, or O.

14. A compound of claim 10 wherein p is 0.

15. A compound of claim 10 wherein R^{a} is selected from the group consisting of haloalkyl, alkyl, substituted alkyl, cycloalkyl, substituted cycloalkyl, aryl, substituted aryl, heteroaryl, substituted heteroaryl, heterocyclic, and substituted heterocyclic.

16. A compound of claim 10 wherein R^{b} is H or methyl.

17. A compound of claim 1 or a stereoisomer, tautomer, or pharmaceutically acceptable salt thereof that is
((R)-2-{(S)-2-[4-(4-Cyclopropylcarbamoyl-pheny)-thiazol-2-ylcarbamoyl]-pyrrolidin-1-yl}-2-oxo-1-phenyl-ethyl)-carbamic acid tert-butyl ester;
(S)-1-((R)-2-Amino-2-phenyl-acetyl)-pyrrolidine-2-carboxylic acid [4-(4-cyclopropylcarbamoyl-phenyl)-thiazol-2-yl]-amide;
Morpholine-4-carboxylic acid ((R)-2-{(S)-2-[4-(4-cyclopropylcarbamoyl-phenyl)-thiazol-2-ylcarbamoyl]-pyrrolidin-1-yl}-2-oxo-1-phenyl-ethyl)-amide;
(S)-4-{(R)-2-[(Morpholine-4-carbonyl)-amino]-2-phenyl-acetyl}-morpholine-3-carboxylic acid [4-(4-cyclopropylcarbamoyl-phenyl)-thiazol-2-yl]-amide;
Morpholine-4-carboxylic acid ((R)-2-{(2S,4S)-2-[4-(4-cyclopentylcarbamoyl-phenyl)-thiazol-2-ylcarbamoyl]-4-hydroxy-pyrrolidin-1-yl}-2-oxo-1-phenyl-ethyl)-amide;
4-Methyl-piperazine-1-carboxylic acid ((R)-2-{(2S,4S)-2-[4-(4-cyclopropylcarbamoyl-phenyl)-thiazol-2-ylcarbamoyl]-4-fluoro-pyrrolidin-1-yl}-2-oxo-1-phenyl-ethyl)-amide;
(S)-3-[(R)-2-(Cyclopropanecarbonyl-amino)-2-phenyl-acetyl]-oxazolidine-4-carboxylic acid {4-[4-(2-morpholin-4-yl-ethylcarbamoyl)-phenyl]-thiazol-2-yl}-amide;
Morpholine-4-carboxylic acid ((R)-2-{(S)-4-[4-(4-cyclopropylcarbamoyl-phenyl-thiazol-2-ylcarbamoyl]-oxazolidin-3-yl}-2-oxo-1-phenyl-ethyl)-amide;
((R)-2-{(S)-4-[4-(4-Cyclopropylcarbamoyl-phenyl)-thiazol-2-ylcarbamoyl]-oxazolidin-3-yl}-2-oxo-1-phenyl-ethyl)-carbamic acid tert-butyl ester;
Morpholine-4-carboxylic acid {(R)-1-cyclohexyl-2-oxo-2-[(S)-4-(4-phenyl-thiazol-2-ylcarbamoyl)-oxazolidin-3-yl]-ethyl}-amide;
Morpholine-4-carboxylic acid {(R)-2-oxo-2-[(S)-4-(4-pyridin-2-yl-thiazol-2-ylcarbamoyl)-oxazolidin-3-yl]-1-thiophen-3-yl-ethyl}-amide;
Morpholine-4-carboxylic acid {(R)-1-cyclohexyl-2-[(2S,4S)-2-(4-furan-2-yl-thiazol-2-ylcarbamoyl)-4-hydroxy-pyrrolidin-1-yl]-2-oxo-ethyl}-amide;
(S)-1-[(R)-2-(Cyclopropanecarbonyl-amino)-2-phenyl-acetyl]-pyrrolidine-2-carboxylic acid [4-(4-cyclopropylcarbamoyl-phenyl)-thiazol-2-yl]-amide;
Morpholine-4-carboxylic acid ((R)-1-benzyl-2-{(2S,4S)-4-hydroxy-2-[4-(4-isopropylcarbamoyl-phenyl)-thiazol-2-ylcarbamoyl]-pyrrolidin-1-yl}-2-oxo-ethyl)-amide;
Piperidine-1-carboxylic acid ((R)-2-{(2S,4S)-2-[4-(4-cyclopropylcarbamoyl-phenyl)-thiazol-2-ylcarbamoyl]-4-fluoro-pyrrolidin-1-yl}-2-oxo-1-pyridin-3-yl-ethyl)-amide;
Morpholine-4-carboxylic acid ((R)-2-{(S)-4-[4-(4-cyclopropylcarbamoyl-phenyl)-thiazol-2-ylcarbamoyl]-oxazolidin-3-yl}-2-oxo-1-phenyl-ethyl)-amide;
(S)-1-[(R)-2-(Cyclopropanecarbonyl-amino)-3-methyl-butyryl]-pyrrolidine-2-carboxylic acid {4-[4-(2-morpholin-4-yl-ethylcarbamoyl)-phenyl]-thiazol-2-yl}-amide;
Morpholine-4-carboxylic acid ((R)-2-{2-[4-(4-cyclopropylcarbamoyl-phenyl)-thiazol-2-ylcarbamoyl]-azetidin-1-yl}-2-oxo-1-phenyl-ethyl)-amide;
((R)-2-{(S)-3-[4-(4-Cyclopropylcarbamoyl-phenyl)-thiazol-2-ylcarbamoyl]-morpholin-4-yl}-2-oxo-1-phenyl-ethyl)-carbamic acid tert-butyl ester;
Morpholine-4-carboxylic acid {(R)-1-cyclohexyl-2-oxo-2-[(S)-2-oxo-5-(4-phenyl-thiazol-2-ylcarbamoyl)-pyrrolidin-1-yl]-ethyl}-amide;
N-Cyclopropyl-6-[2-({(S)-3-[(R)-2-(3,3-dimethyl-ureido)-2-thiophen-3-yl-acetyl]-oxazolidine-4-carbonyl}-amino)-thiazol-4-yl]-nicotinamide;
Morpholine-4-carboxylic acid [(R)-2-[(2S,4S)-4-fluoro-2-(4-furan-2-yl-thiazol-2-ylcarbamoyl)-pyrrolidin-1-yl]-2-oxo-1-(tetrahydro-pyran-4-yl)-ethyl]-amide;
(S)-1-[(R)-2-Cyclohexyl-2-(cyclopropanecarbonyl-amino)-acetyl]-pyrrolidine-2-carboxylic acid [4-(4-cyclopropylcarbamoyl-phenyl)-thiazol-2-yl]-amide;
Morpholine-4-carboxylic acid ((R)-1-{(2S,4S)-4-hydroxy-2-[4-(4-isopropylcarbamoyl-phenyl)-thiazol-2-ylcarbamoyl]-pyrrolidine-1-carbonyl}-2-methyl-propyl)-amide;
Piperidine-1-carboxylic acid ((R)-2-{(2S,4S)-2-[4-(4-cyclopropylcarbamoyl-phenyl)-thiazol-2-ylcarbamoyl]-4-fluoro-pyrrolidin-1-yl}-1-methyl-2-oxo-ethyl)-amide;
((R)-2-{(R)-4-[4-(4-Cyclopropylcarbamoyl-phenyl)-thiazol-2-ylcarbamoyl]-thiazolidin-3-yl}-2-oxo-1-phenyl-ethyl)-carbamic acid tert-butyl ester;
(S)-1-[(R)-2-(Cyclopropanecarbonyl-amino)-3-methyl-butyryl]-pyrrolidine-2-carboxylic acid {4-[4-(2-morpholin-4-yl-ethylcarbamoyl)-phenyl]-thiazol-2-yl}-amide;
1-[(R)-2-(3,3-Dimethyl-ureido)-2-phenyl-acetyl]-azetidine-2-carboxylic acid [4-(4-cyclopropylcarbamoyl-phenyl)-thiazol-2-yl]-amide;
((R)-2-{(S)-3-[4-(4-Cyclopropylcarbamoyl-phenyl)-thiazol-2-ylcarbamoyl]-morpholin-4-yl}-2-oxo-1-methyl-1-phenyl-ethyl)-carbamic acid tert-butyl ester;
(2R,6S)-2,6-Dimethyl-morpholine-4-carboxylic acid ((R)-2-{(S)-2-[4-(4-cyclopropylcarbamoyl-phenyl)-thiazol-2-ylcarbamoyl]-4-methoxy-pyrrolidin-1-yl}-2-oxo-1-phenyl-ethyl)-amide;
6-[2-({(S)-3-[(R)-2-(3-tert-Butyl-ureido)-2-thiophen-3-yl-acetyl]-oxazolidine-4-carbonyl}-amino)-thiazol-4-yl]-N-cyclopropyl-nicotinamide;
(2S,4S)-1-((R)-2-Benzoylamino-2-cyclohexyl-acetyl)-4-fluoro-pyrrolidine-2-carboxylic acid (4-furan-2-yl-thiazol-2-yl)-amide;
(S)-3-[(R)-2-(Cyclopropanecarbonyl-amino)-2-(4-fluoro-phenyl)-acetyl]-thiazolidine-2-carboxylic acid {4-[4-(pyridin-3-ylcarbamoyl)-phenyl]-thiazol-2-yl}-amide;
((R)-2-{(S)-2-[4-(4-Cyclopropylcarbamoyl-phenyl)-thiazol-2-ylcarbamoyl]-piperazin-1-yl}-2-oxo-1-phenyl-ethyl)-carbamic acid tert-butyl ester;
Morpholine-4-carboxylic acid ((R)-2-{(S)-2-[4-(4-cyclopropylcarbamoyl-phenyl)-thiazol-2-ylcarbamoyl]-piperazin-1-yl}-2-oxo-1-phenyl-ethyl)-amide;
Morpholine-4-carboxylic acid ((R)-2-{(S)-2-[4-(4-cyclopropylcarbamoyl-phenyl)-thiazol-2-ylcarbamoyl]-pyrrolidin-1-yl}-1-(4-methyl-thiazol-2-yl)-2-oxo-ethyl]-amide; or
Morpholine-4-carboxylic acid ((R)-2-{(S)-2-[4-(4-cyclopropylcarbamoyl-phenyl)-thiazol-2-ylcarbamoyl]-pyrrolidin-1-yl}-1-(4-methyl-cyclohexyl)-2-oxo-ethyl]-amide.

18. A pharmaceutical composition comprising a pharmaceutically acceptable carrier and a therapeutically effective amount of a compound, stereoisomer, tautomer, or pharmaceutically acceptable salt thereof of claim 1.

19. A method for treating a viral infection in a patient mediated at least in part by a virus in the Flaviviridae family of viruses which method comprises administering to the patient a compound, stereoisomer, tautomer, or a pharmaceutically acceptable salt thereof of claim 1.

20. The method of claim 19 wherein said viral infection is a hepatitis C mediated viral infection.

21. The method of claim 19 in combination with the administration of a therapeutically effective amount of one or more agents active against hepatitis C virus.

22. The method of claim 21 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

23. The method of claim 22 wherein said agent active against hepatitis C virus is interferon.
